(19) United States
(12) Patent Application Publication
(10) Pub. No.: US 2012/0245426 A1
(43) Pub. Date: Sep. 27, 2012

(54) ADJUSTABLE CANNULA

(76) Inventors: Paul L. Salvas, Norton, MA (US); Richard M. Lunn, Kingston, MA (US)

(21) Appl. No.: 13/072,329

(22) Filed: Mar. 25, 2011

(51) Int. Cl. A61B 1/32; A61B 17/34

(52) U.S. Cl. 600/208; 606/108

(57) ABSTRACT

A cannula having a tubular body having a proximal end and a distal end and a central axis defined therethrough, a first protruding member coupled to a first region of the tubular body, and a second protruding member coupled to a second region of the tubular body. Further, the tubular body has a bore formed therethrough. Each of the first protruding member and the second protruding member are coupled to an outer surface of the tubular body. Finally, a length of the tubular body is adjustable along the central axis.

### BACKGROUND OF INVENTION

1. Field of the Invention

Embodiments disclosed herein relate generally to surgical cannulas. Specifically, embodiments disclosed herein relate to an adjustable cannula for effectively engaging a tissue and for establishing a repeatable instrument path within a patient's body.

2. Background

Arthroscopic or, more generally, endoscopic surgical procedures enable closed surgery to be performed via portals through which a variety of elongated instruments may be passed to gain access to an internal surgical work site. Very often, a small incision is made in a patient's tissue, and a disposable cannula is inserted into the incision or portal in order to provide a convenient passageway through which various medical instruments may be passed. A surgeon can then access the region of interest by inserting and manipulating various medical instruments through the open passageway created by the cannula. The action of the surgeon manipulating the instruments may cause frictional forces between the instrument and the cannula, which may cause the cannula to move. The potential exists for the cannula to slip out of the patient's body, requiring the surgeon to stop the current procedure, and re-insert and/or re-position the cannula within the patient's body. This movement of the cannula during a surgical procedure may cause tissue trauma in the area of skin surrounding the cannula. The surgeon performing the procedure may be forced to stabilize the top of the cannula with one hand, while using the other hand to insert the instruments into the cannula.

Additionally, the thickness of tissue which must be traversed by the cannula to access a particular region of the body varies from patient to patient. As such, a variety of different length cannulas are available for use in arthroscopic and laparoscopic procedures, requiring the surgeon or doctor to estimate the thickness of the tissue to be traversed for a particular patient and then select a cannula having the proper length. Further, a portion of the cannula that remains on the outside of the patient's body may extend farther than otherwise desired. Furthermore, because various medical instruments of various lengths may be required for a surgical procedure, an instrument may extend farther than desired from the cannula. On the other hand, the cannula may need to extend into deeper regions of a body cavity, e.g., a joint or an abdominal cavity, to access remote areas of the body.

Accordingly, there exists a need for an adjustable cannula for effectively engaging a tissue and for establishing a repeatable instrument path within a patient's body.

### SUMMARY OF INVENTION

It is an object of the present invention to provide an adjustable cannula for effectively engaging a tissue and for establishing a repeatable instrument path within a patient's body.

According to one aspect of the present invention, there is provided a cannula comprising a tubular body having a proximal end and a distal end and a central axis defined therethrough, in which the tubular body has a bore formed therethrough, a first protruding member coupled to a first region of the tubular body near the proximal end of the tubular body, and a second protruding member coupled to a second region of the tubular body near the distal end of the tubular body, in which each of the first protruding member and the second protruding member are coupled to an outer surface of the tubular body, in which a length of the tubular body is adjustable along the central axis.

According to another aspect of the present invention, there is provided a method of inserting a cannula into a tissue, the method comprising providing a tubular body having a central axis defined therethrough and a bore formed therethrough, in which a first protruding member and a second protruding member are coupled to an outer surface of the tubular body, in which a length of the tubular body is adjustable along the central axis, piercing the outer tissue layer of a body, forming an opening in the outer tissue layer of a body, and inserting the tubular body into the opening in the outer tissue layer of a body and into the tissue.

According to another aspect of the present invention, there is provided a kit for inserting a cannula into a tissue, the kit comprising a tubular body having a proximal end and a distal end and a central axis defined therethrough, in which the tubular body has a bore formed therethrough, a first protruding member coupled to a first region of the tubular body near the proximal end of the tubular body, a second protruding member coupled to a second region of the tubular body near the distal end of the tubular body, in which each of the first protruding member and the second protruding member are coupled to an outer surface of the tubular body, in which a length of the tubular body is adjustable along the central axis, a piercing device configured to pierce an outer tissue layer of a body, and an insertion device having a proximal end and a distal end, the insertion device to be received by the bore of the tubular body.

### BRIEF DESCRIPTION OF DRAWINGS

FIG. 1 is a perspective view of an adjustable cannula in accordance with embodiments disclosed herein.

FIG. 2 is a side view of an adjustable cannula in accordance with embodiments disclosed herein.

FIG. 3 is a flow chart of a method of inserting a cannula into a tissue in accordance with embodiments disclosed herein.

FIG. 4a is perspective view of an adjustable cannula to be received within an opening formed in a shoulder in accordance with embodiments disclosed herein.

FIG. 4b is a side view of an adjustable cannula that is engaged with a layer of tissue of a body in accordance with embodiments disclosed herein.

### DETAILED DESCRIPTION

In one aspect, embodiments disclosed herein relate to an adjustable cannula for effectively engaging a tissue and for establishing a repeatable instrument path within a patient's body. Specifically, embodiments disclosed herein relate to an adjustable cannula having a tubular body having a bore formed therethrough, a first protruding member, and a second protruding member, in which the length of the tubular body is adjustable along a central axis defined through the tubular body.

Embodiments of an adjustable cannula having a tubular body, in which the length of the tubular body is adjustable, disclosed herein may provide a surgical cannula that may be adjusted in length inside of a body. Additionally, the adjustable cannula, according to embodiments disclosed herein, may engage an interior tissue surface of a body as well as an exterior tissue surface of a body in order to effectively engage the tissue and provide stability for the tubular body within a body. Furthermore, the adjustable cannula, according to embodiments disclosed herein, may be able to aid in creating a space between the tissue within a joint to improve visualization and for increased working space during surgery.

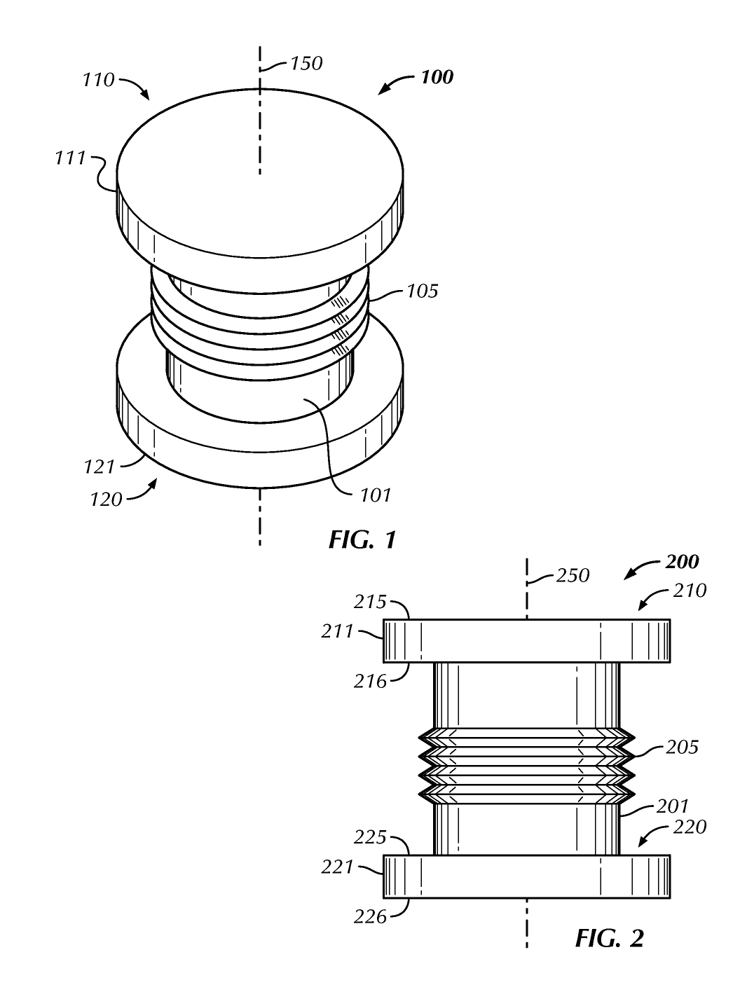

Referring to FIG. 1, a perspective view of an adjustable cannula 100 in accordance with embodiments disclosed herein is shown. The adjustable cannula 100 is an apparatus for effectively engaging a tissue and for establishing a repeatable instrument path within a patient's body and includes a tubular body 101. As used herein, “cannula” may refer to an instrument having a substantially hollow opening for use in a surgical procedure involving disposing the instrument substantially within a body tissue of a human or an animal. As shown, the tubular body 101 has a proximal end 110, a distal end 120, and a central axis 150 defined therethrough. Although the tubular body 101 is shown as a solid, in one or more embodiments, the tubular body 101 may include a bore (not shown) formed therethrough. Further, those having ordinary skill in the art will appreciate that the tubular body may include more or less bores separately formed therethrough than described above. For example, the tubular body may have two, three, four, or more bores formed therethrough to separately receive multiple instruments. As used herein, “bore” may refer to a hole or opening that is formed substantially through the tubular body 101. Those having ordinary skill in the art will appreciate that a bore, according to embodiments disclosed herein, are not limited to holes that are drilled through a tubular body, but also include holes that are formed as part of a tubular body during, for example, an injection-mold process. Further, the tubular body 101 may be made of plastic or any material known in the art that may be deformed or deflected. Furthermore, particular segments of the tubular body 101 may be made of any material known in the art that may be substantially rigid. Although the tubular body 101 shown in FIG. 1 is round, those having ordinary skill in the art will appreciate that the tubular body may be one of a variety of shapes and forms. For example, the tubular body may be square, triangular, hexagonal, or any other shape known in the art.

Referring, still, to FIG. 1, a first protruding member 111 may be coupled to a first region of the tubular body 101 near the proximal end 110 of the tubular body 101. Further, a second protruding member 121 may be coupled to a second region of the tubular body 101 near the distal end 120 of the tubular body 101. As used herein, “coupled” may refer to having two or more elements or components directly attached to each other or indirectly attached to each other. For example, although the first protruding member 111 and the second protruding member 121 are coupled to the tubular body 101, the first protruding member 111 and the second protruding member 121 may not necessarily need to be in direct contact and directly attached with the tubular body 101. As shown, each of the first protruding member 111 and the second protruding member 121 are coupled to an outer surface of the tubular body 101 and may be flexible/deformable. Those having ordinary skill in the art will appreciate that the first protruding member 111 and the second protruding member 121 may be made of plastic or any material known in the art that may be deformed, flexed, or deflected. In one or more embodiments, a diameter of the first protruding member and/or a diameter of the second protruding member may be larger than an outer diameter of the tubular body 101. In one or more embodiments, the outer diameter of the tubular body 101 may be 5-10 mm. However, those having ordinary skill in the art will appreciate that each of the first protruding member 111, the second protruding member 121, the tubular body 101, and the bore (not shown) may have be of any size and may have diameters outside of the range given as an example, above. Those having ordinary skill in the art will also appreciate that the first protruding member 111 and the second protruding member 121 may not be identical. Specifically, although not shown, the first protruding member 111 and the second protruding member 121 may have different diameters, may be made of different materials, and also may have different degrees of flexibility. Further, in one or more embodiments, the first protruding member 111 may be configured to engage with an exterior tissue surface (not shown) of a body (not shown). Furthermore, in one or more embodiments, the second protruding member 121 may be configured to engage with an interior tissue surface (not shown) of a body (not shown). In one or more embodiments, the first protruding member 121 may secure the tubular body 101 to the exterior or outer tissue surface of a body, which may establish a foundation for various medical instruments to be inserted within the bore (not shown) of the tubular body 101 without the aid of a surgeon's other hand to stabilize the tubular body 101. In one or more embodiments, the second protruding member 121 may reduce or prevent encroachment of soft tissue into the working space during surgery, and may help maximize visualization. Further, the second protruding member 121 may secure the tubular body 101 to the interior tissue surface and prevent undesired removal of the tubular body 101 during a surgical procedure.

Further, as shown in FIG. 1, the tubular body 101 includes one or more bellows 105 configured to allow the length of the tubular body 101 to adjust along the central axis 150. In other words, in one or more embodiments, the plurality of bellows 105 may allow the tubular body 101 to compress or extend along the central axis 150. Specifically, the plurality of bellows 105 may allow the tubular body 101 to adjust along the central axis 150 between a first position and a second position. In the first position, the tubular body 101 may be in a fully compressed state and the length of the tubular body 101 along the central axis 150 may be at a minimum. In the second position, the tubular body 101 may be in a fully extended state and the length of the tubular body 101 along the central axis 150 may be at a maximum. Those having ordinary skill in the art will appreciate that other structures allowing a length of a tubular body to adjust along a longitudinal axis may be used. For example, in one or more embodiments, the tubular body 101 may be made of a material, such as an elastomeric plastic or rubber, in which elongation is a material property of the material. Alternatively, in one or more embodiments, the tubular body 101 may include telescoping segments or an internal ratchet-style interface, which may allow the length of the tubular body 101 to be adjustable along the central axis 150. As used herein, “to adjust” may also mean “to be adjusted,” allowing the adjustable cannula 100, according to one or more embodiments, to be automatically adjusted by an automatic adjusting means. For example, the tubular body 101 of the adjustable cannula 100 may be composed of a material that may be adjusted by means of electric current or manipulation by an adjustment device.

Furthermore, although not shown, a seal may be coupled to a region of the tubular body 101 near the proximal end 110 of the tubular body 101. Because many arthroscopic and some laparoscopic procedures require the use of pressurized fluid to distend and irrigate a joint being operated upon, it may be necessary to provide a sealed passageway in order to enable instruments to be passed into and out of the adjustable cannula 100 while maintaining a fluid seal whether or not an instrument (not shown) is in the bore (not shown) of the tubular body 101. As such, in one or more embodiments, the seal may be configured to substantially provide a fluid seal between an interior of the tubular body 101 and an exterior of the tubular body 101. For example, one or more elastomeric membranes may be attached to the proximal end 110 of the tubular body 101. For example, and without limitation, another exemplary seal is shown in U.S. Patent. Publication No. 2008/0294123, which is hereby incorporated herein by reference in its entirety.

Referring now to FIG. 2, a side view of an adjustable cannula 200 in accordance with embodiments disclosed herein is shown. As shown, the adjustable cannula 200 includes a tubular body 201, the tubular body 201 having a proximal end 210, a distal end 220, one or more of bellows 205, and a central axis 250 defined therethrough. As discussed above, although the tubular body 201 is shown as a solid, in one or more embodiments, the tubular body 201 may include one or more bores (not shown) formed therethrough. Further, a first protruding member 211 may be coupled to a first region of the tubular body 201 near the proximal end 210 of the tubular body 201. Furthermore, a second protruding member 221 may be coupled to a second region of the tubular body 201 near the distal end 220 of the tubular body 201. Additionally, as discussed above, the plurality of bellows 205 may allow the tubular body 201 to compress or extend along the central axis 250. Specifically, the plurality of bellows 205 may allow the tubular body 201 to adjust along the central axis 250 between a first position and a second position. In the first position, the tubular body 201 may be in a fully compressed state and the length of the tubular body 201 along the central axis 250 may be at a minimum. In the second position, the tubular body 201 may be in a fully extended state and the length of the tubular body 201 along the central axis 250 may be at a maximum.

As shown, the first protruding member 211 includes a top surface 215 and a bottom surface 216. Likewise, the second protruding member 221 includes a top surface 225 and a bottom surface 226. Those having ordinary skill in the art will appreciate that the adjustable cannula 200 may be rotated 180 degrees, in which a top surface may be known as a bottom surface, and vice versa. In one or more embodiments, once the adjustable cannula 200 is disposed within a body (not shown), e.g., within a joint, the bottom surface 216 of the first protruding member 211 may be used to engage an exterior tissue surface (not shown) of a body. Further, the top surface 225 of the second protruding member 221 may be used to engage an interior tissue surface (not shown) of a body. Once the top surface 225 of the second protruding member 221 is engaged with the inner tissue surface of a body, a surgeon can pull on the tubular body 201 and/or the first protruding member 211 in a direction away from the body in order to enhance joint distension. In other words, once the second protruding member 221 is engaged with the interior tissue surface of a body, the adjustable cannula 200 may be used, e.g., pulled, to manipulate the interior tissue of a body, e.g., a joint, which may promote increased visibility and work space within the body.

The adjustable cannula 200 may be manufactured using a variety of methods and processes. For example, the adjustable cannula 200 may be manufactured using an injection-mold process, in which the tubular body 201 as well as the first protruding member 211 and the second protruding member 221 may be molded using molds filled with a material to form the adjustable cannula 200. Further, the adjustable cannula 200 may be manufactured using a two-step injection-mold process, in which the tubular body 201 of the adjustable cannula 200 may be made of a different material when compared to the first protruding member 211 and the second protruding member 221. For example, in one or more embodiments, the first protruding member 211 and the second protruding member 221 of the adjustable cannula 200 may be made of a hard plastic, in which the tubular body 201 of the adjustable cannula 200 may be made of a more pliable, flexible, elastomeric plastic. Conversely, in one or more embodiments, the tubular body 201 of the adjustable cannula 200 may be made of a hard plastic, in which the first protruding member 211 and the second protruding member 221 of the adjustable cannula 200 may be made of a more pliable, flexible, elastomeric plastic. Those having ordinary skill in the art will appreciate that the adjustable cannula 200 may be composed of a hard or rigid material, a flexible material, or a combination of the two. For example, the adjustable cannula 200 may be composed of a hard plastic or rubber, an elastomeric plastic or rubber, or a combination of the two. In a two-step injection-mold process, a first material that may be used to form the first protruding member 211 and the second protruding member 221 may be first injected into a mold, followed by an injection of a second material that may be used to form the tubular body 201 of the adjustable cannula. Those having ordinary skill in the art will appreciate that the adjustable cannula 200 may be manufactured using other methods commonly known in the art, and that the manufacturing process of the adjustable cannula 200 is not limited to an injection-mold process. For example, the adjustable cannula 200 may be manufactured using a casting process.

A method of inserting a cannula into a tissue, in accordance with embodiments disclosed herein, may include providing a tubular body having a central axis defined therethrough and a bore formed therethrough, providing a piercing device configured to pierce an outer tissue layer of a body, providing an insertion device having a proximal end and a distal end, the insertion device to be received by the bore of the tubular body, piercing the outer tissue layer of a body with the piercing device, forming an opening in the outer tissue layer of a body, disposing the insertion device within the bore of the tubular body, and inserting the tubular body into the opening in the outer tissue layer of a body and into the tissue. A first protruding member and a second protruding member may be coupled to an outer surface of the tubular body. A length of the tubular body may be adjustable along the central axis. The insertion device may be a rod-like member, in which the distal end is a pointed end. Instead, the insertion device may be a rod-like member, in which the distal end is a blunt end. Further, the insertion device may be a device or apparatus configured to fold or angle the second protruding member of the adjustable cannula for insertion into a body. Alternatively, the insertion device may be an obturator. For example, and without limitation, an exemplary obturator is shown in U.S. Patent. Publication No. 2008/0058816, which is hereby incorporated herein by reference in its entirety.

The method may also include adjusting the length of the tubular body between a first position and a second position based on a thickness of the tissue, in which, in the first position, the tubular body is in a fully compressed state and the length of the tubular body is at a minimum, and, in which, in the second position, the tubular body is in a fully extended state and the length of the tubular body is at a maximum. Further, a top surface of the second protruding member may be engaged with an interior tissue surface of a body. Furthermore, a bottom surface of the first protruding member may be engaged with an exterior tissue surface of a body. The method may also include removing the insertion device from within the bore of the tubular body, in which the tubular body remains in the tissue. The method may also include removing the tubular body from the tissue.

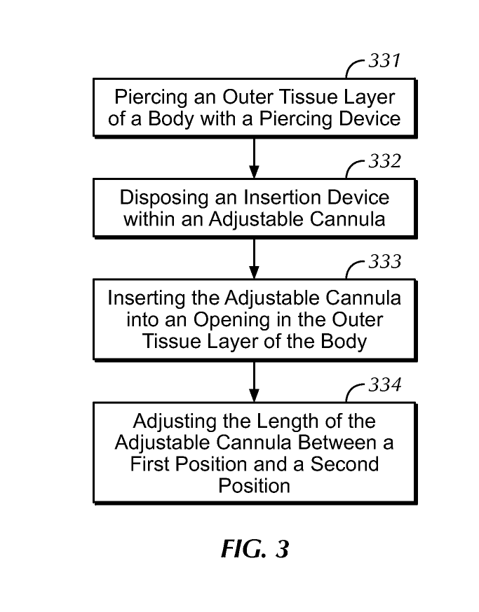

For example, referring to FIG. 3, an incision needle may be used as a piercing device to pierce an outer tissue layer of a body, as shown in step 331. Further, an obturator, as discussed above, may be used as an insertion device and may be disposed within the bore of the tubular body, as shown in step 332. The tubular body may then be guided with the assistance of the obturator into the opening created by the incision needle discussed above, as shown in step 333. Upon insertion into the opening, the second protruding member of the tubular body of the adjustable cannula may be deformed such that the tubular body of the adjustable cannula may traverse through interior tissue of the body to the area of interest within the body, e.g., a joint. Once the adjustable cannula has been positioned within the body, the second protruding member of the tubular body of the adjustable cannula may reform into its original shape and the top surface of the second protruding member may engage with an interior tissue surface of the body. Upon the top surface of the second protruding member of the tubular body of the adjustable cannula engaging with the interior tissue surface of the body, the length of the tubular body may be adjusted between the first position and the second position, such that the bottom surface of the first protruding member of the tubular body of the adjustable cannula is engaged with the external tissue surface of the body, as shown in step 334. Adjusting the adjustable cannula such that the top surface of the second protruding member and that the bottom surface of the first protruding member of the tubular body of the adjustable cannula are engaged with the interior tissue surface and exterior tissue surface of the body, respectively, may provide increase stability for the adjustable cannula within the body. For example, establishing the adjustable cannula, as described above, may prevent a surgeon from having to stabilize the top of the adjustable cannula, e.g. a proximal end of the adjustable cannula, with one hand, while using the other hand to insert the instruments into the adjustable cannula. Further, once the second protruding member is engaged with the interior tissue surface of a body, the adjustable cannula 200 may be used, e.g., pulled, to manipulate the interior tissue of a body, e.g., a joint, which may promote increased visibility and work space within the body.

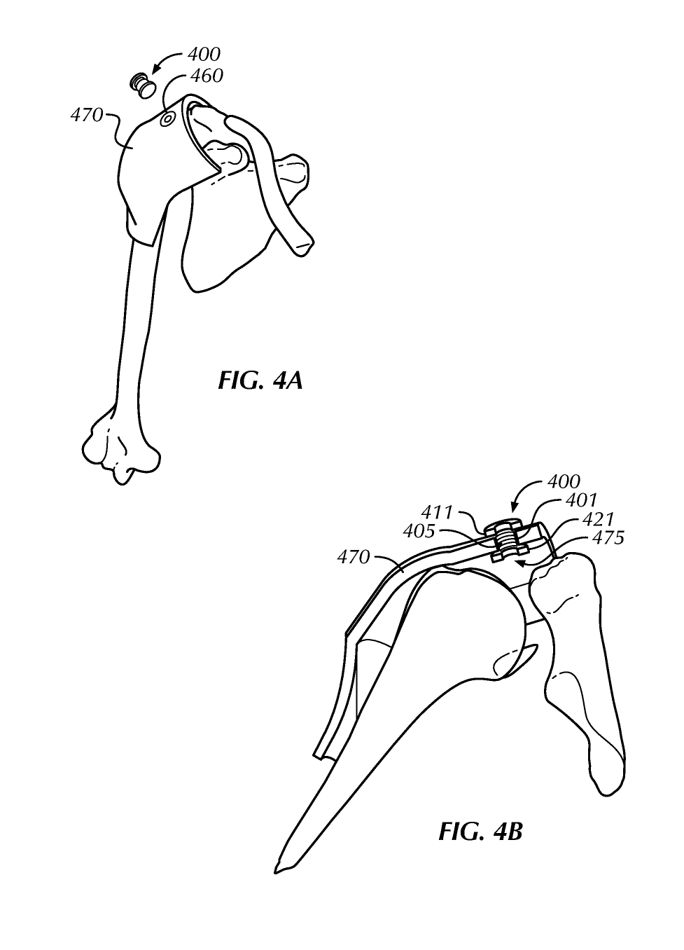

Referring to FIG. 4a, an adjustable cannula 400 to be received within an opening 460 formed in a layer of tissue 470 according to embodiments disclosed herein is shown. As discussed above, the opening 460 may be formed by a piercing device, such as an incision needle, and may be configured to receive the adjustable cannula 400. However, those having ordinary skill in the art will appreciate that a piercing device may include instruments other than an incision needle. For example, the piercing device may be a scalpel or any other surgical instrument capable of piercing a layer of tissue. In one or more embodiments, the opening 460 may be formed through the layer of tissue 470 and may allow the adjustable cannula 400 to form a passage between an exterior and an interior of the layer of tissue 470, e.g., to form a passage to access a joint space. As shown in FIG. 4a, the opening 460 is formed to allow the adjustable cannula to be installed through the epidermis, additional layers of skin, adipose, and muscle to allow the adjustable cannula 400 to access a capsule surrounding a joint. Those having ordinary skill in the art will appreciate that the opening 460 may be formed in a variety of areas on a body to allow the adjustable cannula 400 to access various joint spaces. Although FIG. 4a shows the opening 460 formed through the layer of tissue 470 in the shoulder area, those having ordinary skill in the art will appreciate that an opening may be formed through a layer of tissue on other areas of the body to allow the adjustable cannula 400 to access other particular joint spaces. For example, in addition to a shoulder joint, the adjustable cannula 400 may be used to access a knee or hip joint, or any other joint within a body.

Referring to FIG. 4b, the adjustable cannula 400 is engaged with the layer of tissue 470 according to embodiments disclosed herein is shown. As shown, the adjustable cannula 400 is received within the layer of tissue 470, in which a second protruding member 421 is disposed below the layer of tissue 470, e.g., within the body, and a first protruding member 411 is disposed above the layer of tissue 470, e.g., outside of the body. As discussed above, the layer of tissue 470 may be comprised of, for example, epidermis, additional layers of skin, adipose, and muscle. Further, as discussed above, according to one or more embodiments, the adjustable cannula 400 may include a tubular body 401 having one or more bellows 405 that may allow a length of the tubular body 401 to adjust along a central axis (not shown) of the tubular body 401. As shown, the tubular body 401 extends through the layer of tissue 470 and forms a passage between an exterior of the layer of tissue 470 and a joint space 475. In one or more embodiments, the one or more bellows 405 of the tubular body 401 may allow the length of the tubular body 401 to adjust based on a thickness of the layer of tissue 470. As such, the adjustable cannula 400 may be adjustable or customizable to fit different bodies with tissue layers of varying thickness. As shown, the first protruding member 411 may be engaged with an outer surface of the layer of tissue 470. However, it may not be required for the first protruding member 411 to be physically engaged with an exterior surface of the layer of tissue 470 for the adjustable cannula 400 to be considered to be engaged with the layer of tissue 470. Further, as shown, the second protruding member 421 may not necessarily be physically engaged with the layer of tissue 470. Although it may be beneficial for the second protruding member 421 to be physically engaged with an interior surface of the layer of tissue 470, as discussed above, it may not be required for the second protruding member 421 of the adjustable cannula 400 to be engaged with the layer of tissue 470 for the adjustable cannula 400 to be considered to be engaged with the layer of tissue 470. However, those having ordinary skill in the art will appreciate that the length of the tubular body 401 of the adjustable cannula 400 may be adjusted to allow the first protruding member 411 to engage with an outer surface of the layer of tissue 470, and allow the second protruding member 411 to engage with an inner surface of the layer of tissue 470 when the adjustable cannula 400 is engaged with the layer of tissue 470.

A kit for inserting a cannula into a tissue, in accordance with embodiments disclosed herein, may include a tubular body having a proximal end and a distal end and a central axis defined therethrough, a first protruding member coupled to a first region of the tubular body, a second protruding member coupled to a second region of the tubular body, a piercing device configured to pierce an outer tissue layer of a body, and an insertion device having a proximal end and a distal end, the insertion device to be received by the bore of the tubular body. The tubular body may have a bore formed therethrough. Each of the first protruding member and the second protruding member may be coupled to an outer surface of the tubular body. A length of the tubular body may be adjustable along the central axis.

Advantageously, embodiments disclosed herein may provide an adjustable cannula for effectively engaging a tissue and for establishing a repeatable instrument path within a patient's body. For example, once the adjustable cannula is disposed and effectively engaged within a tissue, as described above, various medical instruments may be inserted and removed from a patient's body while minimizing tissue trauma in the area of skin surrounding the adjustable cannula. Additionally, adjustable cannula, according to embodiments disclosed herein, may provide a fluid seal between the body and the instrument path as well as between the inside and the outside of the body. For example, a first protruding member and a second protruding member, as described above, may form a seal between an exterior tissue surface and an interior tissue surface, respectively. Specifically, the seal formed by the first protruding member and the second protruding member may prevent fluid from escaping from the body during a surgical procedure. Likewise, the seal formed by the first protruding member and the second protruding member may prevent fluid from entering the body during a surgical procedure.

The adjustable cannula, according to embodiments disclosed herein, may also allow a surgeon to adjust the length of the tubular body of the adjustable cannula to accommodate for the tissue thickness of a particular patient. The adjustability of the length of the adjustable cannula may allow surgeons to use the cannula with a variety of patients, varying in size. Further, the adjustability of the length of the adjustable cannula may allow surgeons to use the cannula to access a variety of areas of interest within the body, e.g., various joints as well as various regions of the pelvic area. For example, the adjustable cannula may allow surgeons to use the cannula to access joints, such as a hip or shoulder joint, as well as a variety of sealed organs, such as a kidney or liver. Furthermore, as discussed above, engaging the second protruding member of the tubular body of the adjustable cannula may secure the adjustable cannula to the interior tissue surface and prevent undesired removal of the adjustable cannula during a surgical procedure. Additionally, the first protruding member and the second protruding member of the adjustable cannula may also provide a surface for tissue, e.g., subcutaneous tissue, to compress against. Compressing tissue with the first protruding member and the second protruding member of the adjustable cannula may shorten the pathway that medical instruments may have to travel in order to access areas of interest within the body. Finally, according to embodiments disclosed herein, once the second protruding member is engaged with the interior tissue surface of a body, the adjustable cannula may be used, e.g., pulled, to manipulate the interior tissue of a body, e.g., a joint, which may promote increased visibility, sightline, instrument workability, and work space within the body.

While embodiments have been described with respect to a limited number of embodiments, those skilled in the art, having benefit of this disclosure, will appreciate that other embodiments can be devised which do not depart from the scope of embodiments disclosed herein. Accordingly, the scope of embodiments disclosed herein should be limited only by the attached claims.

What is claimed is:

1. A cannula comprising:
a tubular body having a proximal end and a distal end and a central axis defined therethrough,
wherein the tubular body has a bore formed therethrough;
a first protruding member coupled to a first region of the tubular body; and
a second protruding member coupled to a second region of the tubular body,
wherein a length of the tubular body is adjustable along the central axis.

2. The cannula of claim 1, wherein the tubular body comprises one or more bellows configured to allow the length of the tubular body to adjust along the central axis.

3. The cannula of claim 1, wherein at least one of the first protruding member and the second protruding member are deformable.

4. The cannula of claim 1, further comprising a seal coupled to a region of the tubular body near the proximal end of the tubular body.

5. The cannula of claim 4, wherein the seal is configured to substantially provide a fluid seal between an interior of the tubular body and an exterior of the tubular body.

6. The cannula of claim 1, wherein the tubular body is configured to adjust along the central axis between a first position and a second position.

7. The cannula of claim 6, wherein, in the first position, the tubular body is in a fully compressed state and the length of the tubular body is at a minimum.

8. The cannula of claim 6, wherein, in the second position, the tubular body is in a fully extended state and the length of the tubular body is at a maximum.

9. The cannula of claim 1, wherein the first protruding member is configured to engage with an exterior tissue surface of a body.

10. The cannula of claim 1, wherein the second protruding member is configured to engage with an interior tissue surface of a body.

11. A method of inserting a cannula into a tissue, the method comprising:
providing a tubular body having a central axis defined therethrough and a bore formed therethrough,
wherein a first protruding member and a second protruding member are coupled to the tubular body,
wherein a length of the tubular body is adjustable along the central axis;
piercing the outer tissue layer of a body, forming an opening in the outer tissue layer of a body;
inserting the tubular body into the opening in the outer tissue layer of a body and into the tissue.

12. The method of claim 11, wherein the insertion device is a rod-like member, wherein the distal end of the insertion device is a pointed end.

13. The method of claim 11, wherein the insertion device is a rod-like member, wherein the distal end of the insertion device is a blunt end.

14. The method of claim 11, wherein the insertion device is an obturator.

15. The method of claim 11, further comprising adjusting the length of the tubular body between a first position and a second position based on a thickness of the tissue,
wherein, in the first position, the tubular body is in a fully compressed state and the length of the tubular body is at a minimum, and
wherein, in the second position, the tubular body is in a fully extended state and the length of the tubular body is at a maximum.

16. The method of claim 15, wherein a top surface of the second protruding member is engaged with an interior tissue surface of a body.

17. The method of claim 15, wherein a bottom surface of the first protruding member is engaged with an exterior tissue surface of a body.

18. The method of claim 11, further comprising removing the insertion device from within the bore of the tubular body, wherein the tubular body remains in the tissue.

19. The method of claim 11, further comprising removing the tubular body from the tissue.

20. A kit for inserting a cannula into a tissue, the kit comprising:
a tubular body having a proximal end and a distal end and a central axis defined therethrough,
wherein the tubular body has a bore formed therethrough;
a first protruding member coupled to a first region of the tubular body;
a second protruding member coupled to a second region of the tubular body,
wherein each of the first protruding member and the second protruding member are coupled to an outer surface of the tubular body,
wherein a length of the tubular body is adjustable along the central axis;
one or more devices configured to pierce an outer tissue layer of a body and to be received by the bore of the tubular body.
